(19) United States
(12) Patent Application Publication
(10) Pub. No.: US 2009/0048502 A1
(43) Pub. Date: Feb. 19, 2009

(54) Broadband illuminator for oximetry, hemoglobin, met-hemoglobin, carboxy-hemoglobin, and other blood component monitoring

(76) Inventors: David A. Benaron, Portola Valley, CA (US); Illian H. Parachikov, Belmont, CA (US); Michael R. Fierro, Los Gatos, CA (US)

Correspondence Address: MORGAN, LEWIS & BOCKIUS, LLP., 2 PALO ALTO SQUARE, 3000 EL CAMINO REAL, PALO ALTO, CA, 94306, US

(21) Appl. No.: 12/113,887

(22) Filed: May 1, 2008

Related U.S. Application Data
(63) Continuation of application No. 11/820,809, filed on Jun. 20, 2007.
Application No. 11/820,809 is a continuation-in-part of application No. 11/451,681, filed on Jun. 12, 2006.
Application No. 11/451,681 is a continuation-in-part of application No. 10/651,541, filed on Aug. 29, 2003, now Pat. No. 7,062,306.
Application No. 10/651,541 is a continuation of application No. 10/119,998, filed on Apr. 9, 2002, now Pat. No. 6,711,426.

(51) Int. Cl. A61B 5/1455

(52) U.S. Cl. 600/328

(57) ABSTRACT

An ischemia detection system in two or more somatic measures, collected simultaneously or near-simultaneously, are provided for direct or computational comparison, in which light from light source A (103A) and source B (103B) is detected by a sensor (155), and a difference-weighted value is determined (167), thereby enhancing the value of the spectroscopic measurements over values taken individually and singly.

### CROSS REFERENCE TO RELATED APPLICATIONS

This application is a continuation-in-part of U.S. patent application Ser. No. 11/451,681 filed on Jun. 12, 2006, relating to the detection of local tissue ischemia, which is a continuation-in-part of U.S. patent application Ser. No. 10/651,541 filed on Aug. 29, 2003, now U.S. Pat. No. 7,062,306, which is a continuation of U.S. patent application Ser. No. 10/119,998 filed on Apr. 9, 2002, now U.S. Pat. No. 6,711,426, the disclosures of all of which are incorporated in full by reference.

### FIELD OF THE INVENTION

The present invention relates to devices and methods for providing, simultaneously or near-simultaneously, spectroscopic analysis from more than one somatic site, and more particularly relates to the determination of a difference-weighted analysis wherein the near-simultaneous determination of two (or more) spectroscopically-determined somatic oxygenation saturation values is performed in a manner allowing for the direct and near-simultaneous comparison of these two (or more) somatic saturation values, by direct mutual inspection or computational means, in order to provide synergistic and added medical value above that provided by each individual value considered separately. In another aspect, the present invention provides real-time spectroscopic analysis of in-vivo tissue perfusion from more than one somatic site that is sensitive to local tissue ischemia and insensitive to regional arterial and venous oxygenation.

### BACKGROUND OF THE INVENTION

Ischemia, defined as a reduction in blood flow, can be due to local causes (e.g., due to vascular occlusion or increased metabolism such as a tumor), global causes (e.g., due to body-wide reduced blood flow from reduced cardiac output), or both. However, discriminating the source of changes in tissue oxygenation can be difficult, considering values at each site individually.

Collecting spectroscopic values from two different sites (e.g., organ versus organ, or two sites within the same organ), and considering or analyzing these together as a difference-weighted measure, can add medical value. For example, a growing difference between a stable and normal cheek tissue oximetry, and a falling colon tissue oximetry, points to a colon-centered pathology rather than to a global cause such as impending cardiac failure. Similarly, a widening difference-weighted measurement between a pulse and tissue oximeter (estimates of arterial and venous saturation, respectively), helps pinpoint the source of the change as cardiovascular pathology, rather than increasing pulmonary failure. Last, a widening spatial gradient, such as a difference-weighted value between a pair of sensors that is scanned over a single breast, reduces the noise from organ-wide regional gradients and highlights local inhomogeneities associated with tumors such as breast cancer. Each of these three exemplary difference-weighted values add medical value above what the absolute values, considered alone and separately, would merit.

The noninvasive spectroscopic monitoring of hemoglobin saturation in vivo is known in the art. The great majority of such known devices and methods monitor only at one site (U.S. Pat. No. 6,662,033, WO/2003/003914); such devices do not allow for mutual or computational determination of a difference-weighted value. A few devices and methods in the art teach monitoring at more than one sites. For example, U.S. Pat. No. 6,615,065 describes dual monitoring of the brain, wherein the two sensors are applied to a head of the test subject, taking advantage of the unique hemispheric and non-somatic structure of the brain, to monitor two mutually separate regions within a brain of the test subject, with the two values being simultaneously displayed to allow a user to observationally and mutually compare the two. No computational comparison is taught. Further, the '065 patent teaches that it is the unique, hemispheric structure of the brain that allows the device of '065 to operate, and thus the device would not be suitable for somatic monitoring. In contrast, clinicians recognize that the non-brain (the “somatic”) regional of the body constitute an advantageous early warning system not present in the brain, and are some of the first key tissues to be shut down by the body during impending failure of oxygen delivery to tissue. Similarly, U.S. Patent Application Publication no. 2006/0105319 describes the measuring of two values, arterial and venous. However, again no computational comparison is taught, and one of these values is determined through invasive blood sample, not from spectrophotometric measurement of tissue itself.

All of the above devices are limited to being single measures of oxygenation, are limited or optimized by design or omission to non-somatic tissue, and/or do not allow direct and near-simultaneous mutual comparison or computational processing of at least two somatic values obtained by spectrophotometric measures.

None of the prior devices or methods allow for a difference-weighted spectroscopy that facilitates simultaneous or near-simultaneous comparison of spectroscopic values from two somatic regions or sites by inspection or computation. Such a system has not been previously described, nor successfully commercialized. Thus, further developments are needed.

### SUMMARY AND OBJECTS OF THE INVENTION

The inventors have discovered that certain diseases (vascular ischemia, cancer) are frequently localized, and by comparing at least two somatic values—either multiple sites or times—within the body, resulting in a more sensitive detection of such local conditions.

A salient feature of the present invention is that the detection and treatment of diseases such as somatic ischemia or cancer is aided by use of at least two measurements—either by multiple somatic sensors monitoring at least two nearby or distant regions or by dual measurements made by a single sensor over space or time—allowing a direct comparison of these different spectroscopic values by mutual inspection or computation.

In one aspect, the present invention provides a somatic monitoring apparatus comprising: a first and second sensor, each configured to generate, based upon light produced and/or detected by each sensor, first and second somatic output signals that are a unction of each somatic target site, and a difference unit for comparing said first and second signals, and for generating a difference-weighted output signal based upon this comparison.

In other embodiments, this dual-sensor somatic tissue ischemia monitoring apparatus generates an output signal that is a function of the presence or degree of local tissue ischemia or cancer at a first and second target site, with a display unit configured to display or allow near or substantially simultaneous comparison of said signals at the two target sites. This can be expanded to N sensors, with comparisons of a first through Nth output signals via a difference unit configured to compare at least two of said first through Nth somatic signals, and to generate a difference-weighted output signal based upon said comparison.

In yet another aspect, the difference measurement can be generated using a single sensor moved through space (allowing comparison of two sites with one detector), or used over time (such as reporting changes with time), or even measuring both arterial and tissue oximetry measurements using one probe (allow arteriovenous differences to be detected).

In embodiments of the present invention, we provide both apparatus and methods for the dual, N, and signal sensor approaches. In one embodiment of the invention there is provided a device with dual somatic spectroscopic monitoring sites, including two solid state broadband light sources and sensors for generating, delivering, and detecting light from at least two target sites, for the purpose of allowing a direct comparison of the spectroscopic values by mutual inspection or computation, thereby adding medical value. In another example, the system uses dual phosphor-coated white LED's to produce continuous, broadband, visible light from 400 nm to 700 nm at two somatic sites. Scattered light returning from each target is detected by a wavelength-sensitive detector, and two signals, one from each site, is generated using this wavelength-sensitive information via spectroscopic analysis. The values are displayed or computed in a manner to allow direct comparison of the spectroscopic values by mutual inspection or computation. Systems incorporating the difference-weighted somatic spectroscopic system and medical methods of use are described.

Some embodiments the present invention further provide a device for detecting local ischemia in a tissue at one or more tissue sites, characterized in that the device is configured such that wavelengths of light are selectively emitted, and the selective wavelengths are substantially transmitted through capillaries in tissue while being substantially absorbed by arterial and venous vessels in the tissue.

As will be understood by the detailed description below, the somatic monitoring apparatus provides one or more advantages. For example, by way of illustration and in no way limiting the invention, one advantage is that the system and method may be constructed to detect ischemia, cancer, or changes in perfusion.

Another exemplary advantage is that a physician or surgeon can obtain improved real-time feedback regarding local tissue ischemia, cancer, or perfusion in high-risk patients, and to respond accordingly.

Another exemplary advantage is that ischemia (low delivery of oxygen to tissues) can be differentiated from pulmonary-induced hypoxemia (low arterial saturation).

Yet another exemplary advantage is that local changes in oximetry (vascular disease) can be differentiated from mixed or global changes (low cardiac output).

Another advantage is that the detector of the present invention may be actively coupled to a therapeutic device, such as a pacemaker, to provide feedback to the pacing function, or passively coupled to a therapeutic device, such as applied to a stent to monitor stent performance over time, based upon the detection and degree of local ischemia. Ischemia sensing may be used to enable detection of many types of disease, such as tissue rejection, tissue infection, vessel leakage, vessel occlusion, and the like, many of which produce ischemia as an aspect of the disease.

The breadth of uses and advantages of the present invention are best understood by example, and by a detailed explanation of the workings of a constructed apparatus, now in tested in human subjects. These and other advantages of the invention will become apparent when viewed in light of the accompanying drawings, examples, and detailed description.

### BRIEF DESCRIPTION OF THE DRAWINGS

The breadth of uses and advantages of the present invention are best understood by example, and by a detailed explanation of the workings of a constructed device. These and other advantages of the present invention will become apparent upon consideration of the following detailed description, taken in conjunction with the accompanying drawings, in which like reference characters refer to like parts throughout, and in which:

FIG. 1 a schematic diagram of a difference-weighted spectroscopy system incorporating a white LED and constructed in accordance with embodiments of the present invention;

FIG. 2 shows a medical monitor system constructed in accordance with embodiments the present invention;

FIG. 3A shows a pulsatile broadband signal intensity using a single probe monitor constructed to monitor both arterial and capillary saturation in accordance with some embodiments of the present invention;

FIG. 3B shows a peak systolic and trough diastolic pulse oximetry signal measured using a single probe difference monitor constructed in accordance with some embodiments of the present invention; and

FIG. 4 shows an exemplary sensor probe having one light and two (dual) monitoring fibers for monitoring two closely located sites, in this case located at different depths in a tissue, according to some embodiments of the present invention.

### DETAILED DESCRIPTION OF EMBODIMENTS OF THE INVENTION

### Definitions

For the purposes of this invention, the following definitions are provided for illustration purposes. These definitions are not intended to limit the scope of the invention:

Head or Cranial: Associated with the Head or Skull, respectively, as opposed to the body tissue (c.f., Somatic, below). Stedman's Medical Dictionary, 27th edition, states that cranial is “Relating to the cranium or head.” Blood perfusion to the brain and head, via the carotid supply, can be very different than to somatic tissues, such as liver, intestine, heart, kidney, and others.

Somatic: Tissue in the body and central organs, as opposed to the brain (c.f., brain). Stedman's Medical Dictionary, 27th edition, states that this is “[r]elating to the soma or trunk”. Organs within the body are considered somatic tissues, and include the liver, spleen, intestine, heart, kidney, muscle, and pancreas. Oxygenation and measures in somatic tissues are central to monitoring for sufficiency of oxygen delivery to tissue in the body as a whole.

Ischemia: A condition in which the perfusion of a tissue is locally inadequate to meet its metabolic needs. Ischemia is distinguished from low blood flow per se in that low blood flow alone does not guarantee ischemia (such as during tissue cooling on which flow can be low without significant ischemia), nor does high flow rule out or prevent ischemia (such as during sepsis or when the blood delivered does not contain adequate oxygen). Ischemia is a co-existing condition in many different types of illnesses, including infection (sepsis), tissue rejection (host vs. graft disease), heart attack (myocardial ischemia), stroke (cerebral ischemia), acute or chronic organ failure, diabetic peripheral vascular disease, and other conditions.

Perfusion: The flow of blood or other perfusate per unit volume of tissue, as in ventilation/perfusion ratio. Reduction in perfusion is a major clinical problem, and it is associated with, but not equivalent to, ischemia.

Difference-Weighted: A measurement that is formed from the direct or indirect comparison of two or more oxygenation values, such as somatic venous saturation at organ A to somatic venous saturation of organ B. Another difference measurement is the difference between arterial and venous saturation such as described in detail in co-pending U.S. patent application Ser. No. 11/451,681. Another difference measure is the comparison of a measured value to a baseline or historical value.

Spectroscopy. Measurement of material, including tissue, using light. Such measures can involve a spectrum composed of only a few wavelengths, such as two discrete wavelengths, or can involve a spectrum recorded over a range using a broadband light source, and a wavelength-resolved detector.

One embodiment of the device will now be described. This device has been built in prototype form, tested in the laboratory under experimental conditions, and tested on animals under Animal Study Review Board approval, as shown in some of the data which follow the initial description of one embodiment of the system.

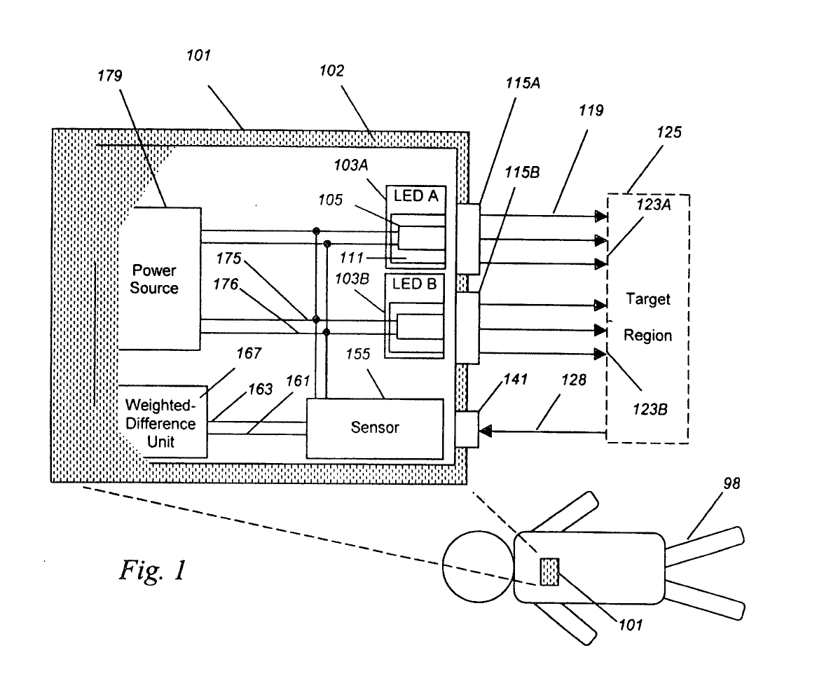

A cut-away schematic showing the interior of spectroscopic device or apparatus 101 according to embodiments of the present invention is shown in FIG. 1. Device 101 is preferably surrounded by soft silicone exterior shell 102, permitting a good grip while scanning device 101 across a target region, or for implantation for chronic monitoring. Typically, exterior shell 102 is constructed from approved Class VI biocompatible materials as recognized by the U.S. FDA or other medical device regulatory agencies. Portions of sensor 155, power source 179, light source LED A 103A and LED B 103B, or other components may protrude as needed from this shell within the spirit of this invention, provided that the protruding parts themselves are biocompatible as required.

Within device 101, source LED 103A is illustrated in its component parts. Broad spectrum white light is emitted by a high conversion-efficiency white LED 105 (e.g., The LED Light, model T1-3/4-20W-a, Fallon, NV). Source 105 is itself embedded into a plastic beam-shaping mount using optical clear epoxy 111 to allow light generated in diode 105 to be collimated, thus remaining at a near-constant diameter after passing through optical window 115A to leave device 101. Light then is able to pass forward as shown by light path vectors 119, with at least a portion of this light optically coupled to first target region 123A in target 125. Note that while target region 125 may be in some instances a living tissue, the tissue itself is not considered to be a claimed part of this invention.

A portion of the light reaching region 123A of target 125 is backscattered and returns as to device 101, as shown by light path vectors 128, to optical collection window 141. Collection window 141 in this embodiment is a glass, plastic, or quartz window, but can alternatively be merely an aperture, or even be a lens, as required. Light then strikes sensor 155, where it is sensed and detected.

Similarly, within device 101, there is a second light source, LED 103B is illustrated in its component parts, constructed in much the same manner as LED 103A, however light this time exits by optical window 115B, to strike second target region 123B in target 125. Again, a portion of the light reaching region 123B is backscattered and returns to device 101 via light path vector 128, to optical collection window 141, striking sensor 155.

Sensor 155 may be comprised of a number of discrete detectors configured to be wavelength-sensitive, or may be a continuous CCD spectrometer, with entry of light by wavelength controlled by gratings, filters, or wavelength-specific optical fibers. In any event, sensor 155 transmits an ischemia signal related to the detected light backscattered from target 125, producing an electrical signal sent via wires 161 and 163 to the unit that determines a weighted difference, difference unit 167.

Light source 103A and 103B could be instead multiple, with up to N light sources, constructed as described, or in a varying manner. In any event, Light source 103A and 103B also has two electrical connections 175 and 176, connecting light sources 103A and 103B to power source 179. In this embodiment, power source 179 is an inductive power supply, capable of receiving an inductive field from externally powered coil and RFID receiver. Such coils and receivers are well known.

Operation of the device may now be described.

Device 101 is scanned across a breast, for example in a patient being screened for breast cancer. The device may measure the various components of the breast such as lipid and water, and/or it may measure tissue hemoglobin saturation. It may be placed on the breast directly, or it can be placed at a distance. In the latter case, vectors 119 are fiber optics extended from device 101 and into close proximity to the target heart muscle, sufficient for optical coupling. Then the patient is allowed to heal after surgery, and the implantable device is left inside the patient's body, without a direct physical connection to the outside world.

In this example, device 101 is normally powered down and in a resting (off) state. At some point, it is desired to test the target heart muscle for the presence of ischemia. Power source 179 located within device 101, produces sufficient power for device 101 to power up and turn on. Light sources 103A, 103B, and others if present, begin to illuminate the target 125, in this case heart muscle. Sensor 155, which is an embedded spectrophotometer, receives backscattered light, resolves the incoming light by wavelength, a marker of ischemia. Under control of lines 175 and 176, LED 103A is first scanned, with an estimated tissue saturation (as determined by tissue oximeters arranged as known in the art, for example, the commercially available T-Stat model 303 Tissue Oximeter may be used, whose design and methods are incorporated into this specification by reference) of 72%. Next, under control of lines 175 and 176, LED 103B is illuminated, producing an estimated tissue saturation of 72%. There values are sent to difference Unit 167, and the difference is found to be zero, which is the median value one expects in normal tissue without cancer.

Once the measurement is completed, device 101 powers down and returns to a resting state.

In an alternative embodiment, power source 179 may be charged during proximity to external coil, or have an internal battery source, allowing device 101 to operate when external coil 179 is not present. Difference unit 167 may then transmit without being directly queried, such as in response to a dangerous level of ischemia.

The breadth of uses and the basis of the present invention is best understood by example, and thus the detailed description will be further illustrated by the following examples. These examples are by no means intended to be inclusive of all uses and applications of the apparatus, merely to serve as a case study by which a person, skilled in the art, can better appreciate the methods of utilizing, and the scope of, such a device.

### EXAMPLE 1

### Simultaneous Two-Site Two-Organ Somatic Difference Monitoring

In this example, a clinical application related to ischemia is described. Here, a surgeon is repairing the aorta. There are several reasons why the local tissue oxygenation may fall. For example, the patient is under anesthesia, and a general depression (reduction) of cardiac output may occur. If so, the delivery of oxygen to all parts of the body will fall. On the other hand, if the blood vessel supplying the colon, which arises in part from the aorta, is occluded, then the saturation to the colon will fall, but not the saturation to the cheek. Therefore, by looking at the saturation of both the cheek and colon at substantially the same time, or by displaying a difference between the two values, the cause of the drop in local oxygenation may be determined to be either local and due to the vascular repair (e.g., large difference, in this case the absolute value of |Δ saturation|>10%) which is an indication of local ischemia, or systemic and due to hypotension or cardiac failure (e.g., small difference, in this case the absolute value of |Δ saturation|<10%), which is an indication of systemic ischemia

This is shown in the following table:

TABLE 1
The difference (Δ) between check and colon oxygenation
is small (|<10%|) under normal conditions, and during system-
wide, whole-body, global reductions in heart output, hematocrit, or
oxygenation from the lungs. In contrast, a large difference between
check and colon oxygenation (|>10%|) is a sign of disparate flow,
and likely of local ischemia.

| | Cheek | | | |
|---|---|---|---|---|
| | (Buccal) | Gut (Colon) | Δ | Local |
| Site | Oxygenation | Oxygenation | Cheek-Colon | Ischemia? |
| Normal | 76% | 71% | +5% | No |
| Low Heart Output | 42% | 48% | −6% | No |
| Bad Colon Artery | 76% | 22% | +44% | YES |

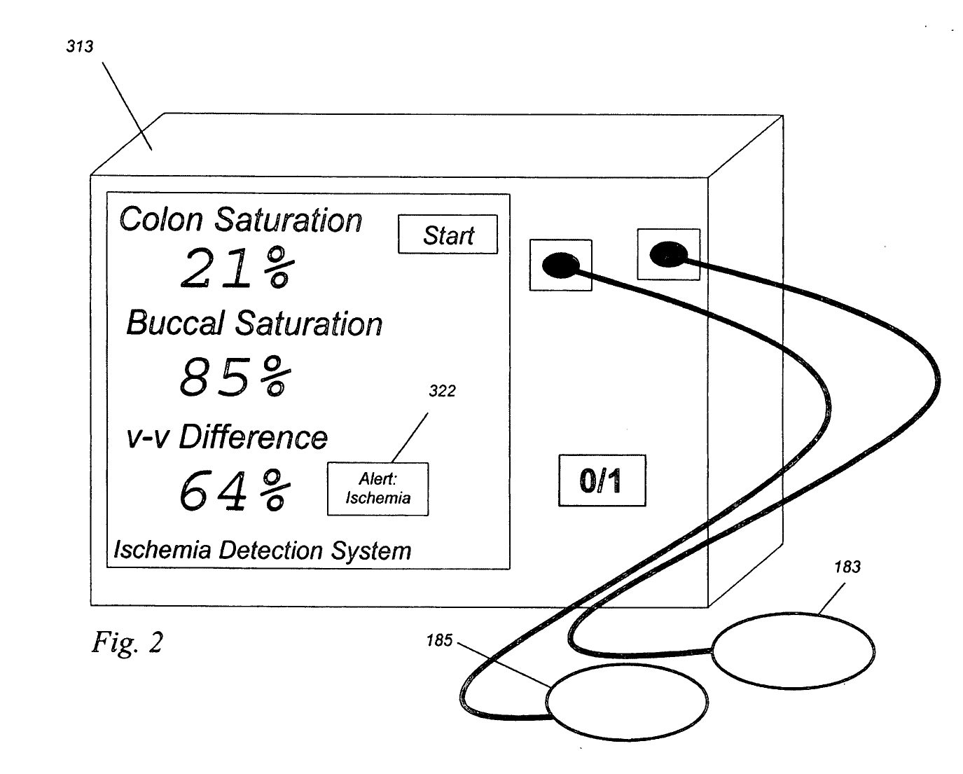

A device displaying two values, simultaneously or near-simultaneously measured, as well as a difference-weighted value display, is shown in FIG. 2 according to some embodiments of the present invention. Monitor or display 313 has two somatic probes 183 and 185 attached, each placed at difference sites. This number of probes could, for other embodiments, be any number of N probes, where N is two or more, within the spirit of the invention. Monitor 313 displays the results of these two sites of measurement, as well as a veno-venous (or Δ) difference of 64%. In other embodiments, the display of N values itself allows a user to manually and directly compare the two values, adding medical value, or alternatively, only the difference-weighted value alone could be displayed, within the spirit of the invention. In view of this large, calculated veno-venous difference, alert 322 is displayed to the user.

Note that near-simultaneous display of the measurement of two or more somatic sites, in this case somatic tissue oxygenation as compared at two sites using a dual-site somatic tissue oximeter constructed in accordance with the present invention, allows either a direct, mutual comparison by an observer of these two displayed values, or a calculation or computation, and then display of, this difference-weighted value. Each of these, dual display for direct, mutual inspection, or calculation of a processed, weighted difference, can be a useful difference-weighted measurement. Further, it is noted that this difference-weighted value is inherently advantageous, adding medical value and relevance to either value taken alone and singly, such as by allowing detection of a local or regional ischemia with better precision, or faster recognition of an ischemic event, or by allowing more rapid identification of the source (cardiac/pulmonary) of the low oxygenation, among advantages illustrated herein. Other advantages, not discussed here, may be learned, and are incorporated into the broad list of medical advantages intended within the scope of the present invention. It is not intended that the medical advantages be subject to limitation by omission of such additional advantages.

### EXAMPLE 2

### Simultaneous Two-Site Single-Organ Somatic Difference Monitoring

In the example above, two different organs were studied. In this example, the monitoring of a single organ, the breast, is described. It is toward this Example that the embodiment of FIG. 1 is directed.

In breast cancer, the detection of angiogenesis, the proliferation of new blood vessels, is a key feature of cancer that lets the cancer gain the ability to grow and spread. However, the background variation in blood content in the breast between women of different ages and breast composition makes the use of a single-site blood-content threshold less useful than it could otherwise be. That is, the range of normal blood content in breast tissue between different women is so large that the increase in blood due to cancer can be lost in that broad range.

To illustrate this, consider data from women with breast cancer. By looking at the difference measurement of the oxygenation at one location on the breast as compared to another near-simultaneously or simultaneously measured point of the breast, and by displaying this difference, local tumor ischemia can be detected to be present (large local difference, in this case the absolute value of Δ saturation>10%) or not present (small difference, in this case the absolute value of Δ saturation<10%), as shown:

TABLE 2
The difference in oxygenation between two nearby regions of the
human breast is small under normal circumstances. A tumor
produces a local region of a high gradient of change in oxygenation
(and also in deoxyhemoglobin content). This difference can be
lost in the local variations (sites A and B, two sites within each
region), but there is a large difference that is a sign of a tumor
when one sensor is near the tumor, and the other
is actually over the tumor.

| | Breast Site A | Breast Site B | Δ Site | Local |
|---|---|---|---|---|
| Site | Saturation | Saturation | A − B | Ischemia? |
| Normal 1 | 76% | 74% | +2% | No |
| Normal 2 | 71% | 68% | +3% | No |
| Normal 3 | 63% | 66% | −3% | No |
| Tumor 4 | 78% | 66% | +12% | YES |

This Site A vs. Site B comparison gains utility because the local variations in oxygenation within a region (at two sites) are small, but the variations between patients is large. In Error! Reference source not found. 2, the range of normals above is 15%, but by looking at differences between sites, only one patient is seen to have cancer.

### EXAMPLE 3

### Multi-Site Single-Organ Somatic Difference Monitoring

In the above example, pairs of data were taken, one pair at a time. In this example, instead of plotting values from a single pair, embodiments of the present invention provide for plotting real time difference values from many measures at many sites.

Again, using data from human subjects with and without breast cancer, the following table can be generated. Such differences can be found by having a difference in spatial separation at two points, as shown as the difference (delta) values at 5 sites labeled A-E on each subject, as follows:

TABLE 3
The Spatial difference at multiple sites by plotting differences,
reduces the noise in breast tissue saturation, and allows simple
detection of tumor near site C of Patient Tumor 4, in which the
saturation difference has a negative then positive deflection
(or vice-versa) during scanning.

| Patient | Δ Site A | Δ Site B | Δ Site C | Δ Site D | Δ Site E |
|---|---|---|---|---|---|
| Normal 1 | 2% | 4% | −3% | 5% | −3% |
| Normal 2 | 0% | −2% | 3% | 2% | −3% |
| Normal 3 | 1% | 4% | −1% | −4% | −1% |
| Tumor 4 | −1% | −4% | −18% | 13% | 3% |

Alternatively, the above differences can be found by a single emitter/detector pair that is scanned over the tissue. Using a 3-D positional sensor (X-Y-Z) or 2-D surface motion sensor (such as the motion detection pad from an optical mouse, based upon a LED and CCD to detect translation across a surface), measures can be taken a multiple real-time instances during motion, and the delta value calculated from the different positions of the detector. So, at time zero there is no delta, while at time 1 the delta is the time 1 value minus the time 0 value, at time 2 the delta is the time 2 value minus time 1, and so on.

### EXAMPLE 4

### Difference Abdominal Monitoring for Necrotizing Colitis Detection

In this example, the monitoring of the premature newborn abdomen is described. A baseline probe is placed over another tissue, such as the buccal mucosa.

As a probe is scanned across the abdomen of normal infants and across one with a regional portion of bowel with low oxygenation, the following table is created:

TABLE 4
The difference display allows the values abnormal for the oxygenation
status to show ischemic necrotizing enterocolitis at sites C and D of
patient Ischemia 4 to be displayed and/or detected.

| Patient | Δ Site A | Δ Site B | Δ Site C | Δ Site D | Δ Site D |
|---|---|---|---|---|---|
| Normal 1 | −4% | 3% | −3% | −6% | 1% |
| Normal 2 | 0% | 6% | 2% | −4% | −2% |
| Normal 3 | −4% | 0% | 2% | 3% | 5% |
| Ischemia 4 | 3% | 5% | −22% | −37% | −10% |

In each of these cases, the medical accuracy and value of these measurement comes from or is enhanced by the simultaneous measurement of two or more somatic sites.

It goes without saying that other configurations and embodiments shall fall within the spirit of the invention, provided that two or more measures in the body are provided more or less simultaneously. For example, the reverse situation, in which one or more sensors and a single light source is used is well within the spirit of the invention, as are multiple sensors and multiple sources, provided that more than one location is measured more or less contemporaneously, to allow an enhanced value from simultaneous measures.

Last, an advantage is simply that the user can use one monitor at multiple sites, without having to purchase multiple monitors.

### EXAMPLE 5

### Single or Dual Site Arterio-Venous Difference Monitoring

In prior examples, venous or tissue oxygenation values were compared. In this example, arterial and venous values are compared according to another aspect of the present invention.

We have shown that the difference between a pulse oximeter and a tissue oximeter, one showing arterial and the other showing venous saturation, allows ischemia (low tissue oxygen delivery) and hypoxemia (low arterial blood saturation) be distinguished as described in more detail in co-pending parent application U.S. Ser. No. 11/451,681, the entire disclosure of which is hereby incorporated by reference. Embodiments of the present invention employ this difference arterial and venous saturation into a real-time calculation, and make it possible for real-time monitoring previously not available.

In the table below, values of tissue and arterial values measured in animals are summarized. By making this a real-time calculation, these values could be demonstrated in real time, rather than determined after the fact, as had been performed in these earlier data:

TABLE 5
The difference display allows the differences, here calculated
after the fact by separate measures, to be displayed. Values
for Normoxia, Hypoxemic Hypoxia, and Ischemic Hypoxia (low
flow and delivery) to be distinguished in animal and human
models (from Benaron et al, Anesthesiology, 2004).

| | Normoxia | Hypoxemic Hypoxia | Ischemic Hypoxia |
|---|---|---|---|
| Subject | (Δ saturation %) | (Δ saturation %) | (Δ saturation %) |
| Human | 21-29% | 16% | 51-91% |
| Animal | 25-28% | 22-38% | 66-83% |

In this example, this table can be incorporated into monitor 313 of FIG. 2, in which the difference value of 64% is used to turn on ischemic hypoxia alert 322. Again, by making this a real-time calculation, these values could be demonstrated in real time, rather than determined after the fact, as had been performed in these earlier data.

In some embodiments, a device is provided with dual somatic spectroscopic monitoring sites where light sources and sensors generate and detect light from at least two tissue target sites and are configured to emit light at selective wavelengths where the selective wavelengths are substantially transmitted through capillaries in tissue while being substantially absorbed by arterial and venous vessels in the tissue. This aspect is described in detail in co-pending U.S. patent application Ser. No. 11/451,681 filed on Jun. 12, 2006, the entire disclosure of which is hereby incorporated by reference. More specifically, in some embodiments the device of the present invention is configured to operate at a wavelength range, such as a range of 400 to 600 nm, and more specifically blue to green visible illuminating light (at around 500 nm). The inventors have discovered that this range of wavelengths penetrates larger vessels very poorly while being relatively highly transmitted by the capillaries, thus allowing sensitivity of the ischemia measurement at the two or more tissue sites to be increased. This is wavelength range is taught away from by oximetry art, which instead is focused on the advantages of near infrared light. This locally-weighted and microvascular-weighted measurements to detect ischemia in a local portion of a target tissue site may be utilized to determine the difference in measurements between two or more somatic monitoring sites. A locally-weighted measurement, as used herein, is a measurement that is weighted toward the condition of a local tissue near a sensor probe, rather than the blood flowing in the larger vessels that is not in physiological contact, e.g., capable of direct and significant oxygen exchange, with that local tissue. A microvascular-weighted measurement is a measurement that is weighted toward the smallest vessels, such as those having 20 microns or smaller, rather than to the blood flowing in the larger vessels that is not in physiologic contact with the local tissue.

Due to the deep penetration of large vessels by infrared (and red) light, using infrared or red light to measure light transmittance and absorbance through tissue reflects a wide range of vessel sizes and results in measurements that are not substantially locally-weighted or microvascularly-weighted. In contrast, a blue-green weighted measurement penetrates larger vessels poorly but capillaries well, and does not travel to sufficient depths that would force inclusion of many large vessels. That is, using blue-green light to measure light transmittance and absorbance through tissue results in a substantially locally-weighted and microvascular-weighted measurement. This is non-obvious and counterintuitive to the prior art, which tends to teach the use of infrared light for its tissue-penetrating ability and against the use of the shallow-penetrating blue end of the visible spectrum.

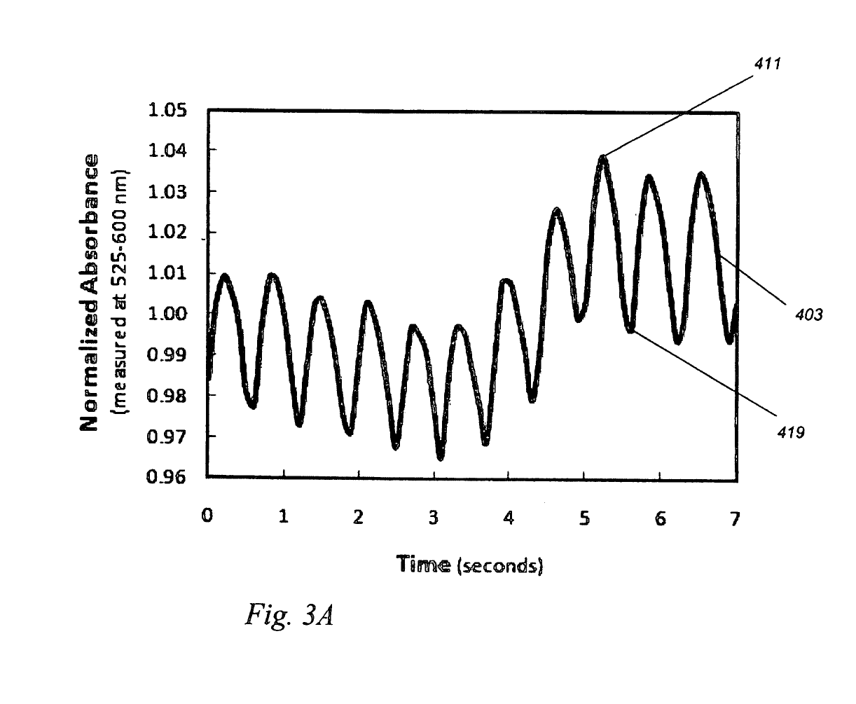

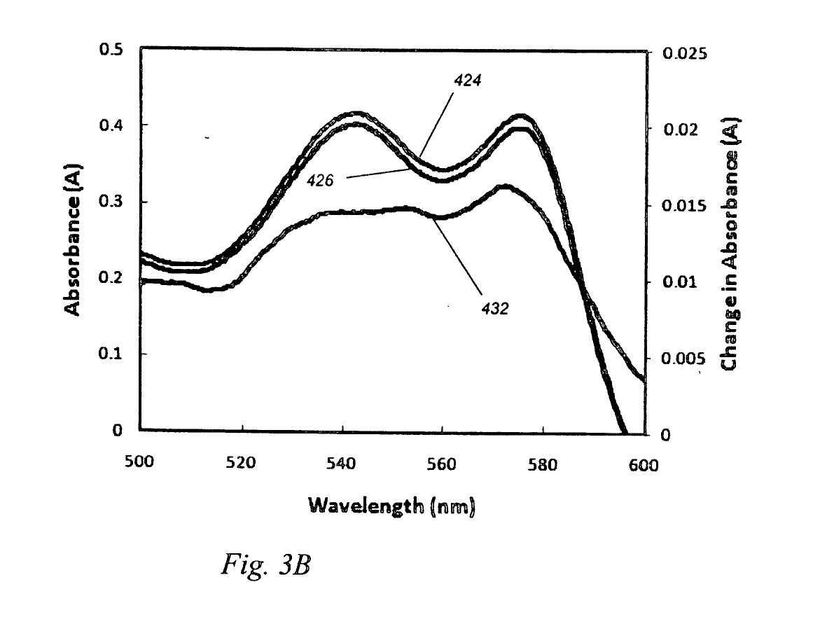

Another aspect of the arterial-venous approach is that it can be performed using the present invention, in the absence of a pulse oximeter, but with the a dual or single site multispectral or broadband tissue oximeter alone. This was first measured by one of the inventors in the present invention in the 1990's, and has now been further developed and an enabling embodiment invented using the device as disclosed in the present invention, with measurement even using a single probe over time produces multispectral pulse oximetry plethysmograph 403, as reflected in data collected from a human subject in FIG. 3A. The intensity of the signal changes for a wide range of wavelengths over time, between a minimum to a maximum intensity, in a pulsatile manner. The maximum absorbance occurs during the period the tissue is most filled with blood (usually near the peak of systolic arterial blood pressure, but sometimes associated with the transmitted pressure of a ventilator breath, or other blood volume changes), which corresponds to local pulsatile absorbance maximum 411. Similarly, as the tissue blood content falls, there is a minimum absorbance during the period the tissue is least filled with blood (usually near the end of the diastolic arterial blood pressure resting phase, but sometimes associated with the release of pressure of a ventilator breath, or other changes), which corresponds to local pulsatile absorbance minimum 419.

The important issues of the combined measurement of the pulse and tissue oximetry signals here are several-fold. First, by measuring both the venous and the arterial signal, the difference measurement can be obtained using a single probe, or by two tissue oximetry probes, wherein the arterial pulsations can be analyzed using conventional or proprietary pulse oximetry techniques (computer analysis of the difference signal, ratios at wavelengths, or even using self-adjusting variable-weight signal extraction technologies). Such a difference spectrum is illustrated for broadband pulse oximetry in FIG. 3B, where systolic peak absorbance signal 424 and diastolic trough absorbance signal 426 can be subtracted to produce delta signal 432. Delta signal 432 may then be further analyzed to determine an arterial saturation estimate. Unsubtracted peak absorbance signal 424 and diastolic trough absorbance signal 426 can then be analyzed (separately or as an average) to yield a conventional tissue capillary oximetry signal, as disclosed in this invention. The difference weighted measure here is then the arterial minus the venous signal, as described earlier in this example.

The ability to generate a perfusion measurement warrants some attention here. The magnitude of variation in with time of delta signal 432 (either in absolute terms, as a fraction of the total hemoglobin signal, or as a volume-corrected signal) can be used as a perfusion index. Another measure of perfusion is the A−V difference itself, which given a fixed amount of oxygen extraction by the tissue, widens as the inverse of the A−V (or pulse minus tissue) difference. For example, if the perfusion falls in half, and the arterial saturation is 100%, one would expect the tissue saturation to fall from 70% (30% difference) to 40% (60% difference, or twice 30%), in the absence of other physiological corrections. Combination of magnitude of time-varying delta signal 432 and A−V difference measures, additionally even including other measures such as laser Doppler capillary velocity that are known in the art or correction of these signals for blood volume determined optically, could be used to generate a more accurate or robust perfusion index, all optically determined or even augmented with other flow-sensitive methods such as ultrasound Doppler.

### EXAMPLE 6

### Layer-Stripping Difference Monitoring for Colon Ischemia

In the prior examples, oxygenation values were compared using a simple subtraction. In this example embodiments of the present invention provide an apparatus or device comprising a probe with a single light source and two detection fibers at different distances is used to monitor colon during interventional surgery. Alternatively, the apparatus may be comprised of a probe with two light sources and one detection fiber, or separate detection fibers and separate light sources. Other arrangements may be used by those of skill in the art, all of which are within the spirit of the present invention.

When colon or intestine is joined at surgery, the joined site is called the anastomosis. Leakage at the joining site, called anastomotic leakage, occurs after surgery in 5%-14% of patients undergoing esophageal, gastric, intestinal, and colon anastomosis, typically several days to weeks after surgery. Leakage results in gut and colon contents spilling into normally sterile body cavities, and results in prolonged hospitalizations, sepsis, and death. However, it is currently not predictable at the time of surgery which patients will go on to leak, preventing additional and known steps to be taken in the operating room that could help avoid future leakage.

A high-specificity mucosal, intraoperative ischemia detection system would permit real-time detection of patients at risk for leakage, allowing for real-time surgical attempts at correction of the problem. Leakage is, of course, multi-factorial, but the cause of a leak is frequently local ischemia caused by poor local perfusion, difficult access with insufficient “good” bowel to sew to, preexisting infection, and difficult location that leads to poor local perfusion. These each lead in turn leads to breakdown and leakage at the site of anastomosis. By identifying the subset of patients with poor perfusion and likely leak, those patients would be able to be the focus of more invasive procedures, procedures that would not be justified if used in all patients, but certainly justified in patents at high risk for leak.

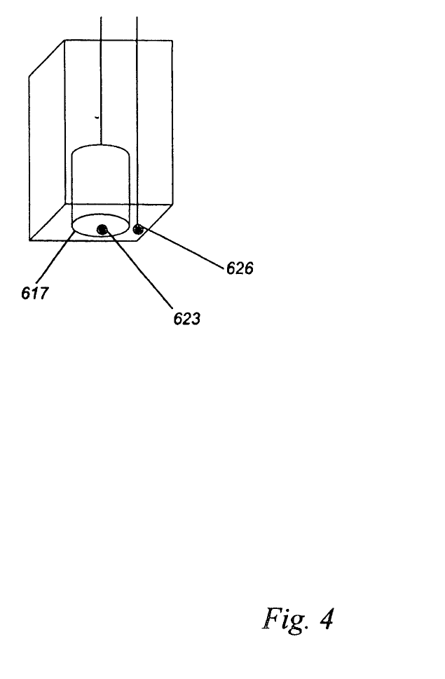

We tested the ability of this system to detect colon ischemia, and found that in open surgery, the top few millimeters oxygenate from the air, even if the gut is truly ischemic. Therefore we constructed a scanner, such as that shown in FIG. 1, in which optical illumination occurs at two difference locations, and measurement is made through one fiber. Equivalently, one light could be used, with two different measurement fibers, as shown in FIG. 4. Here, light source 617 contains central light detection fiber 623, as well as peripheral light detection fiber 626.

Using the device as constructed in FIG. 4, as attached to monitor 313 of FIG. 2, spectra were collected at two separations, and then the saturation was deduced using a standard radiological approach called layer stripping, in which the effect of the overlying layer is removed from the underlying layer. In this embodiment, monitor 313 comprises a difference unit programmed with software know in the art for performing layer stripping. In this approach, it is not the saturation values that are subtracted, but rather by collecting and mathematically removing the narrowly-spaced spectrum (collected from light source 617 and central fiber 623) from the spectrum collected from the more widely spaced pair (light source 617 and peripheral fiber 626), a common data analysis tool called layer stripping in radiology, and then reanalyzing the remaining spectrum for oxygen saturation, deeper ischemia in the breast or other target tissue can reliably be detected, as shown:

TABLE 6
The difference, in this case calculated by removing the spectra
collected from the deeper-collected spectrum, and then
reanalyzing the values, allows the deeper oxygenation
to be determined, thus showing tissues which
may not heal in anastomosis

| | Narrow | Deep | Color | Deep |
|---|---|---|---|---|
| Actual Tissue Measured | Pair Only | Pair Only | Difference | Ischemia? |
| Ischemia Under | 80% | 40% | 09% | Yes |
| Oxygenated Surface | | | | |
| Normal Under Ischemic | 45% | 62% | 69% | No |
| Surface | | | | |
| Normal Tissue Under | 70% | 65% | 63% | No |
| Normal Mucosa | | | | |

In patients with ischemia, the surgical procedure can then be changed by this value, and conversely those with normal values may be allowed to undergo higher risk procedures. For example, if the ischemic site is the anastomosis of two regions of a colon, and the saturation is low, then the tissue should not be sewn together, as it will not heal. One may also use this approach to study the effect of surgical staples on ischemia, in order to determine that surgical staple lines are too tight to heal well.

We have discovered a dual or multiple somatic measurement difference method that allows for more sensitive detection of local ischemia and or local cancer using oximetry measurements. As described above, in some embodiments the apparatus comprises two phosphor-coated LED's and integrated collimating optics constructed in accordance with the present invention to produce light at two or more target sites. Light backscattered by each target site is collected by the same or multiple sensors, allowing for an index or measure of ischemia to be determined, and subsequently transmitted to a comparison unit that additional compares the two results. This device has immediate application to several important problems, both medical and industrial, and thus constitutes an important advance in the art.

We claim:

1-23. (canceled)

24. A broadband method of spectroscopic monitoring in vivo comprising the steps of:
(a) placing an oximeter probe on, in or near a tissue site;
(b) illuminating the tissue site with broadband illumination from a broadband solid-state illumination source having a usable spectral bandwidth of at least 100 nm;
(c) monitoring broadband light returning from a monitored site; and,
(d) determining a measure of hemoglobin oxygenation or other hemoglobin, tissue, or blood measures based upon said broadband light.

25. The method of claim 24 wherein said tissue site is selected from the list of tissue sites consisting of oropharynx, nasopharynx, esophagus, stomach, duodenum, ileum, colon, skin, and other tissue sites.

26. The method of claim 24 wherein said probe is selected from the list of probes consisting of probes for medical endoscopic use, targetable injection needles, catheters, needles, catheters with extendable needles, nibblers, devices with jaws, scissors, probes that measure colon oxygenation, probes that measure by pulse oximetry, probes that measure arterial oxygenation, probes that measure oxygen delivery to the body's core organs, probes that measure in the gastrointestinal system at or in the oropharynx, nasopharynx, esophagus, stomach, duodenum, ileum, colon, or other gastrointestinal tissues.

27. A broadband method of spectroscopic monitoring in vivo comprising the steps of:
(a) illuminating a tissue site with a broadband solid-state illumination source having at least a visible light portion with some wavelengths shorter than 700 nm;
(b) monitoring at least a portion of the broadband light returning from the tissue site; and,
(c) determining an index of hemoglobin or other blood components based upon said returning light.

28. The method of claim 27 wherein said visible portion of broadband illumination comprises a portion of the visible spectral band from 500 nm to 600 nm.

29. The method of claim 24 wherein said step of determining comprises determining an oxygenation index over time during a medical intervention, wherein said intervention is further modified based upon the result of said step of determining.

30. The method of claim 29 wherein said medical intervention is a vascular procedure directed toward a coronary blood vessel, an arterial blood supply, or other blood vessels, said tissue site is the gastrointestinal mucosa, and said oxygenation index is a measure of ischemia.

31. The method of claim 29 wherein said medical intervention is a vascular tie off or other vascular procedure during which the local blood flow may change.

32. A broadband optical spectroscopy system comprising:
(a) a broadband, solid-state illumination source for illuminating a target region having at least a visible light portion with at least some wavelengths shorter than 700 nm;
(b) an optical collector for collecting broadband light returning from said target region and for transmitting a collected signal to a spectroscopic monitor; and
(c) oximetry monitoring means for receiving and analyzing collected broadband light as a function of wavelength, said receiving and analyzing including at least a portion of received visible light, and for determining an oxygen saturation index of hemoglobin in said target region based upon said monitored visible light.

33. A broadband optical spectroscopy system comprising:
(a) a broadband, solid-state illumination source, said source having a usable spectral bandwidth of at least 100 nm for illuminating a target region;
(b) an optical collector for collecting broadband light returning from said target region and for transmitting a collected signal to a spectroscopic monitor; and,
(c) spectroscopic monitoring means for receiving and analyzing collected broadband light, and for determining an index or measure of hemoglobin or other blood components in said target region based upon said monitored light.

34. The oximeter system of claim 32 or 33 wherein said saturation or index determination is performed at least in part using collected light in at least a portion of the visible violet to green spectrum from 400 to 600 nm.

35. The oximeter system of claim 32 or 33 wherein said saturation or index determination is performed at least in part using collected light in at least a portion of the green to orange spectral band from 500 to 600 nm.

36. A broadband spectroscopy method for monitoring in vivo comprising the steps of:
(a) illuminating a tissue with a broadband solid-state illumination source having a usable spectral bandwidth of at least 100 nm;
(b) monitoring broadband light returning from a monitored site, wherein the monitoring includes the step of monitoring an AC component of broadband light returning from a monitored tissue site; and,
(c) determining a pulse oximetry hemoglobin oxygenation index, or a measure of met-hemoglobin, carboxy-hemoglobin or other blood components, based upon said monitoring, where the step of determining is based at least in part upon said AC component of the returning light.

37. The method of claim 24, 27 or 36 wherein the broadband illumination source is a broadband LED.

38. The method of claim 37 wherein the broadband LED is a white LED

39. The method of claim 37 wherein the broadband LED is comprised of multiple discrete LEDs.

40. The method of claim 39 wherein the multiple LEDs operate in at least one wavelength band.

41. The method of claim 37 wherein the broadband illumination source is comprised to multiple light emitting elements, such as a combination of different light emitting diodes, to produce a spectrum of light.

42. The method of claim 24 or 27 wherein the step of monitoring includes the step of monitoring an AC component of broadband light returning from a monitored tissue site; and wherein the step of determining includes the step of determining a pulse oximetry hemoglobin saturation or a measure of met-hemoglobin, carboxy-hemoglobin or other blood components, based at least in part upon said AC component of the returning light.

43. The method of claim 27 wherein said broadband source emits useable light over a wavelength range of at least 100 nm or more.

44. The method of claim 37 wherein the broadband LED contains a blue LED and a phosphor.

45. The method of claim 37, wherein the broadband LED contains an LED a fluorescent dye.

46. The method of claim 36 wherein the broadband source operates to produce at least a portion of its illumination in the infrared spectrum.

47. The method of claim 36 wherein the broadband source operates to produce at least a portion of its illumination in the ultraviolet spectrum.

48. The method of claim 36 wherein a target signal is enhanced, produced, or detected, at least in part, by one or more of the following methods: light absorbance, polarization, optical rotation, scattering, fluorescence, Raman effects, phosphorescence, fluorescence decay, re-emission, use of a contrast agent, dye-shift, or other known spectroscopy techniques.

49. The method of claim 36 wherein said determining involves a solution of multiple simultaneous spectroscopic equations.

50. The system of claim 32, 33, or 36 wherein said system is selected from the group of systems consisting of spectrophotometers, oximeters, systems that perform pulse oximetry, microdevices, a microchip, a lab-on-a-chip, or other small optical device with space and size constraints, disposable optical devices, or other optical spectroscopy devices and systems.

51. The system of claim 33 or 36 wherein the system is a medical device selected from the group of devices consisting of probes for medical endoscopic use, targetable injection needles, catheters, needles, catheters with extendable needles, nibblers, devices with jaws, scissors, probes that measure colon oxygenation, probes that measure by pulse oximetry, probes that measure arterial oxygenation, probes that measure oxygen delivery to the body's core organs, probes that measure in the gastrointestinal system including could reasonably include the oropharynx, nasopharynx, esophagus, stomach, duodenum, ileum, colon, or other gastrointestinal tissues.

52. The system of claim 32 or 33, wherein the system is a medical device configured for use in oximetry.

53. The system of claim 52 wherein the system is medical device is an oximeter probe.

54. The system of claim 52 wherein the system is a medical device configured for use in monitoring met-hemoglobin, carboxy-hemoglobin, and other blood components.

55. The method of claim 36 wherein the broadband source light is pulsed.

56. The method of claim 36, wherein the broadband source light is analyzed as time-resolved, frequency-resolved, or spatially-resolved.

57. The system of claim 52 wherein the broadband source is incorporated into a pulse oximeter.

58. The system of claim 32 or 33 wherein the monitoring comprises monitoring an AC component of broadband light returning from a monitored tissue site; and wherein the determining comprises determining a pulse oximetry hemoglobin saturation or a measure of met-hemoglobin, carboxy-hemoglobin or other blood components, based at least in part upon said AC component of the returning light.
